(19) United States
(12) Patent Application Publication
(10) Pub. No.: US 2004/0167524 A1
(43) Pub. Date: Aug. 26, 2004

(54) Anti-splay medical implant closure with central multi-surface insertion and removal aperture

(76) Inventor: Roger P. Jackson, Prairie Village, KS (US)

Correspondence Address: John C. McMahon, PO Box 30069, Kansas City, MO, 64112, US

(21) Appl. No.: 10/783,272

(22) Filed: Feb. 20, 2004

Related U.S. Application Data
(63) Continuation-in-part of application No. 10/236,123, filed on Sep. 6, 2002, now Pat. No. 6,726,689.

(51) Int. Cl. A61B 17/58

(52) U.S. Cl. 606/73

(57) ABSTRACT

An anti-splay closure with a multi-surfaced aperture, such as a multi-lobular socket, includes a cylindrical body with an external, continuous, helically extending anti-splay guide and advancement flange. The multi-lobular socket includes a plurality of circumferentially spaced lobes extending parallel to a closure axis of the plug and which circumferentially alternate with spline receiving grooves extending parallel to the closure axis. The closure is used with an open headed bone implant screw having arms that are provided with internal, helically extending mating guide and advancement structures complementary to the body flange to allow slidable mating with the body upon rotation thereof and radial interlocking between the arms and the body.

### Cross-Reference to Related Application

This is a continuation-in-part of co-pending U.S. patent application Ser. No. 10/236,123 filed Sep. 6, 2002 for HELICAL WOUND MECHANICALLY INTERLOCKING MATING GUIDE AND ADVANCEMENT STRUCTURE, which is now U.S. Pat. No. ______ .

### BACKGROUND OF THE INVENTION

The present invention is directed to a closure for use in closing between spaced arms of a medical implant and securing a rod to the implant. In particular, the closure includes a non-circular multi-surfaced or multi-lobular internal bore for improved engagement by a complementary shaped tool for purposes of insertion and removal, and an interlocking helical guide and advancement structure that prevents splaying of upper ends of walls of the implant within which the closure is placed away from an axis of rotation of the closure.

Medical implants present a number of problems to both surgeons installing implants and to engineers designing them. It is always desirable to have an implant that is strong and unlikely to fail or break during usage. It is also desirable for the implant to be as small and lightweight as possible so that it is less intrusive on the patient. These are normally conflicting goals and often difficult to resolve.

One particular type of implant presents special problems. In particular, spinal bone screws, hooks, etc. are used in many types of back surgery for repair of injury, disease or congenital defect. For example, spinal bone screws of this type are designed to have one end that inserts threadably into a vertebra and a head at an opposite end. The head is designed to receive a rod or rod-like member in a channel in the head in which the rod is both captured and locked to prevent relative movement between the various elements subsequent to installation. The channel in the head is open ended and the rod is simply laid in the open channel. The channel is then closed with a closure member. The open headed bone screws and related devices are much easier to use and in some situations must be used instead of closed headed devices.

While open headed devices are often necessary and often preferred for usage, there is a significant problem associated with them. In particular, the open headed devices conventionally have two upstanding arms that are on opposite sides of a channel that receives the rod member. The top of the channel is closed by a closure after the rod member is placed in the channel. The closure can be of a slide in type, but such are not easy to use. Threaded nuts are sometimes used that go around the outside of the arms. Such nuts prevent splaying of the arms, but nuts substantially increase the size and profile of the implant which are not desirable. Many open headed implants are closed by plugs, bodies or closures that screw into threads between the arms, because such have a low profile. However, threaded plugs have encountered problems also in that they produce radially outward directed forces that lead to splaying or spreading of the tops of the arms or at least do not prevent splaying caused by outside forces that in turn loosen the implant. In particular, in order to lock the rod member in place, a significant force must be exerted on the relatively small plug. The tightening forces are required to provide enough torque to insure that the rod member is clamped or locked in place relative to the bone screw, so that the rod does not move axially or rotationally therein. Torques on the order of 100 inch-pounds are typical.

Because open headed implants such as bone screws, hooks and the like are relatively small, the arms that extend upwardly at the head can rotate relative to the base that holds the arms so that the tops of the arms are rotated or bent outward relatively easily by radially outward directed forces due to the application of substantial forces required to secure the rod member. Historically, early closures were simple plugs that were threaded with V-shaped threads and screwed into mating threads on the inside of each of the arms. But, as noted above, conventional V-shaped threaded plugs tend to splay or push the arms radially outward upon the application of a significant amount of torque, which ends up bending the arms sufficiently to allow the threads to loosen or disengage and the closure to fail. To counter outward directed application of forces, various engineering techniques were applied to resist the spreading forces. For example, the arms were significantly strengthened by substantially increasing the width of the arms. This had the unfortunate effect of substantially increasing the weight and the profile of the implant, which was undesirable.

The tendency of the open headed bone screw to splay is a result of the geometry or contour of the threads typically employed in such devices. In the past, most bone screw head receptacles and screw plugs have employed V-shaped threads. V-threads have leading and trailing sides oriented at angles to the screw axis. Thus, torque on the plug is translated to the bone screw head at least partially in an axial outward direction, tending to push or splay the arms of the bone screw head radially outward. This in turn spreads the internally threaded receptacle away from the thread axis so as to loosen the plug in the receptacle. The threads also have smooth or linear surfaces in a radial direction that allow slippage along the surfaces since they at best fit interferingly with respect to each other and have in the past not interlocked together. Thus, forces other than insertion forces can act to easily splay the arms since the surfaces slide rather than interlock.

The radial expansion problem of V-threads due to the radial outward component of forces applied to a V-thread has been recognized in various types of threaded joints. To overcome this problem, so-called “buttress” threadforms were developed. In a buttress thread, the trailing or thrust surface is oriented perpendicular to the thread axis, while the leading or clearance surface remains angled. This theoretically results in no radially inward or outward directed forces of a threaded receptacle in reaction to application of torque on the threaded plug. However, the linear surfaces still allow sideways slippage, if other forces are applied to the arms.

Development of threadforms proceeded from buttress threadforms, which in theory have a neutral radial force effect on the screw receptacle, to reverse angled threadforms, which theoretically positively draw the threads of the receptacle radially inward toward the thread axis when the plug is torqued. In a reverse angle threadform, the trailing side of the external thread is angled toward the thread axis instead of away from the thread axis, as in conventional V-threads. While buttress and reverse threadforms reduce the tendency to splay, the surfaces are not interlocking and the arms can still be bent outward by forces acting on the implant. The threads can be distorted or bent by forces exerted during installation. Therefore, while these types of threadforms are designed to not exert radial forces during installation, at most such threadforms provide an interference or frictional fit and do not positively lock the arms in place relative to the closure plug.

Furthermore, it is noted that plugs of this type that use threadforms are often cross threaded. That is, as the surgeon tries to start the threaded plug into the threaded receiver, the thread on the plug is inadvertently started in the wrong turn or pass of the thread on one arm. This problem especially occurs because the parts are very small and hard to handle. When cross threading occurs, the plug will often screw part way in the receiver and then “lock up” so that the surgeon is led to believe that the plug is properly set. However, the rod is not secure relative to the bone screw or other implant and the implant fails to function properly. Therefore, it is also desirable to have a closure that resists cross threading in the receiver.

As stated above, it is desirable for medical implants to have strong and secure elements which are also very lightweight and low profile so that the overall implant impacts as little as possible upon the patient. However, strong and secure are somewhat divergent goals from the goals of lightweight and low profile. Thus, size, weight., and profile must all be taken into consideration and minimized, as much as possible, consistent with effective functioning.

In order to provide sufficient strength and friction to resist movement of the various elements once the closure plug is seated, it is necessary to apply a fairly substantial amount of torque to the closure.

### SUMMARY OF THE INVENTION

The present invention provides a closure for use particularly with an open-headed bone implant screw to secure another implant structural member therein. The closure has a cylindrical plug, base or body. A non-circular multi-surfaced bore or aperture extends axially through or partly through the body and is accessible from a trailing surface of the body to form a structure or mechanism for engagement by an installation and/or removal tool of similar cross section to install or remove the body from the bone screw, if necessary. As used herein, the term multi-surfaces is intended to include multi-lobular or any other horizontal cross section (relative to the drawings) that is not round and that is adapted to mate with an insertion tool or removal tool, so as to provide grip or purchase to the tool while the tool rotates about an axis of rotation of the closure so as to operably install and set the closure or alternatively to remove the closure upon reverse rotation of the tool. In particular, within the body of the closure, the removal aperture is formed into a non-round multi-surfaced socket to receive a closure removal tool having a non-round cross sectional shape which is complementary to the shape of the socket. As noted above, the socket has a horizontal cross section or footprint that is non-round so that after a tool of similar cross section is placed in the aperture, an interference fit is provided when the tool is rotated, so as to rotate the body.

The multi-surfaced socket of the aperture is preferably formed by a plurality of centrally facing surfaces positioned circumferentially about a socket axis and extending generally parallel to the axis that is coaxial with an axis of rotation of the body. Such surfaces may include a plurality of planar surfaces, such as or similar to a hexagonal Allen socket or non-planar surfaces, including or similar to Torx (trademark of Textron, Inc.) or other multi-lobular shapes. A multi-lobular shape preferably includes a plurality of circumferentially spaced, centrally facing, rounded lobes separated by axial grooves or channels which receive splines of the closure removal tool. The splines of the removal tool are circumferentially spaced and separated by axially extending, rounded, outwardly facing concave grooves which are shaped to closely engage the lobes of a matingly shaped closure socket. The shapes of the closure socket and closure removal tool provide for positive, non-slip engagement of the removal tool with the closure body while avoiding the localized concentrations of stresses which can occur with other configurations of separable torque transfer arrangements.

The closure is also provided with a non-threaded guide and advancement structure for securing the closure in a receiver and locking the arms against splaying once the closure is seated in the implant. Preferably, the receiver is a rod receiving channel of an open-headed bone screw, hook or other medical implant in which the channel has an open top and is located between two spaced apart arms forming the open head of the bone screw.

The body of the closure is cylindrical and has an external guide and advancement flange extending helically about the body, relative to the body axis of rotation. The guide and advancement flange preferably has a compound, anti-splay type of contour which cooperates with complementary internal mating guide and advancement structures formed into the inner surfaces of spaced apart arms forming the open head of the bone implant screw. The flange has such a compound contour that includes an inward anti-splay surface component on the flange which faces generally inward toward the body axis. The mating guide and advancement structures of the bone screw head have a complementary contour to the body flange including outward anti-splay surface components which face outward, generally away from the body axis.

The inward anti-splay surface component is preferably formed by an enlarged region near an outer periphery of the body flange near a crest of the flange. The outward anti-splay surface components are formed near an outer periphery of the mating guide and advancement structures by enlargement thereof. The complementary anti-splay surface components of the closure and head slidably engage upon rotation and cooperate to interlock the body with the arms so as to resist splaying tendencies of the arms when the closure is strongly torqued or when other forces are applied to the various elements thereof.

In use, the closure and open-headed bone screw are used to anchor a spinal fixation member, such as a rod, by threadedly implanting the bone screw into a bone and clamping the rod within the head of the bone screw using the closure body. In order to enhance clamping engagement of the rod, the body may be provided with structural features which cut into the surface of the rod to thereby reduce the likelihood of translational or rotational movement of the rod relative to the bone screw. The body is preferably provided with a “cup point”, set ring, or V-ring on a forward end of the body to cut into the surface of the rod when the body is tightly torqued into the head of the bone screw. In some embodiments, the body is also provided with a central axial point on the leading end thereof.

### OBJECTS AND ADVANTAGES OF THE INVENTION

Therefore, objects of the present invention include providing an improved closure for use with an open headed bone screw; providing such a closure having a cylindrical base or body that provides a low or minimized profile subsequent to installation of the closure; providing such a closure having removal structure enabling positive, non-slip engagement of the closure by a removal tool; providing such a closure having an axially extending multi-surfaced aperture that opens onto the trailing surface of the body for use in both installing and removing the body; providing such a closure having such an aperture that forms a removal tool receiving socket including a plurality of centrally facing surfaces positioned circumferentially about an axis of rotation of the body and extending generally along the axis to form the non-round, non-slip socket to receive a removal tool having a complementary shape; providing such a closure which has such a removal aperture with a multi-lobular shape including a plurality of circumferentially spaced, centrally facing, rounded lobes separated by axial grooves or channels which receive splines of the removal tool; providing such a closure in combination with an open headed bone implant screw for use in anchoring a bone fixation structural member, such as a rod; providing such a combination in which the open headed bone screw includes a pair of spaced apart arms forming a rod receiving channel; providing such a combination including an external guide and advancement flange on the closure body and internal mating guide and advancement structures located on inner surfaces of the bone screw head which slidably mate upon rotation of the body and that interlock and cooperate to resist tendencies of the arms to splay or diverge when the closure is torqued tightly into clamping engagement with a rod positioned in the channel or when external forces are applied to the implant; providing such a combination including elements to enhance setting engagement of the closure body with a rod in the bone screw channel; providing such a combination in which a forward end of the closure body is provided with an axially aligned point and/or a peripheral cup point or V-ring to cut into the surface of the rod when the body is torqued and tightened, to resist translational and rotational movement of the rod relative to the bone screw; and providing such an anti-splay closure body with a multi-surface aperture which is economical to manufacture, which is secure and efficient in use, and which is particularly well adapted for its intended purpose.

Other objects and advantages of this invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is an enlarged perspective view of an anti-splay closure with a multi-surfaced removal aperture in accordance with the present invention.

FIG. 2 is a side elevational view of the closure at a further enlarged scale.

FIG. 3 is a top plan view of the closure and illustrates details of the multi-surfaced aperture of the closure.

FIG. 4 is a bottom plan view of the closure and illustrates a V-ring on a forward end of the closure.

FIG. 5 is a cross sectional view of the closure, taken on line 5-5 of FIG. 3, and illustrates internal details of the multi-surfaced aperture of the closure.

FIG. 6 is a fragmentary side elevational view at a reduced scale of the closure in combination with an open headed bone screw implant in a vertebra with the closure partially installed in the implant.

FIG. 7 is a view similar to FIG. 6 and illustrates full installation of the closure into the implant.

FIG. 8 is an enlarged cross sectional view of the body of the present invention positioned in clamping relationship within an open headed bone screw and illustrates details of an anti-splay guide and advancement structure of the body and bone screw head.

FIG. 9 is an enlarged top plan view of the closure within the open headed bone screw.

FIG. 10 is an enlarged perspective view of a second embodiment of an anti-splay closure with a multi-surfaced removal aperture in accordance with the present invention.

FIG. 11 is a side elevational view of the second closure at a further enlarged scale.

FIG. 12 is a top plan view of the second closure and illustrates details of the multi-surfaced aperture of the closure.

FIG. 13 is a bottom plan view of the second closure and illustrates a V-ring on a forward end of the closure.

FIG. 14 is a cross sectional view of the second closure, taken on line 5-5 of FIG. 3, and illustrates internal details of the multi-surfaced aperture of the second closure.

FIG. 15 is a fragmentary side elevational view at a reduced scale of the second closure in combination with an open headed bone screw implant in a vertebra with the second closure partially installed in the implant.

FIG. 16 is a view similar to FIG. 6 and illustrates full installation of the second closure into the implant.

FIG. 17 is an enlarged cross sectional view of the body of the second closure positioned in clamping relationship within an open headed bone screw and illustrates details of an anti-splay guide and advancement structure of the body and bone screw head.

FIG. 18 is an enlarged top plan view of the closure within the open headed bone screw.

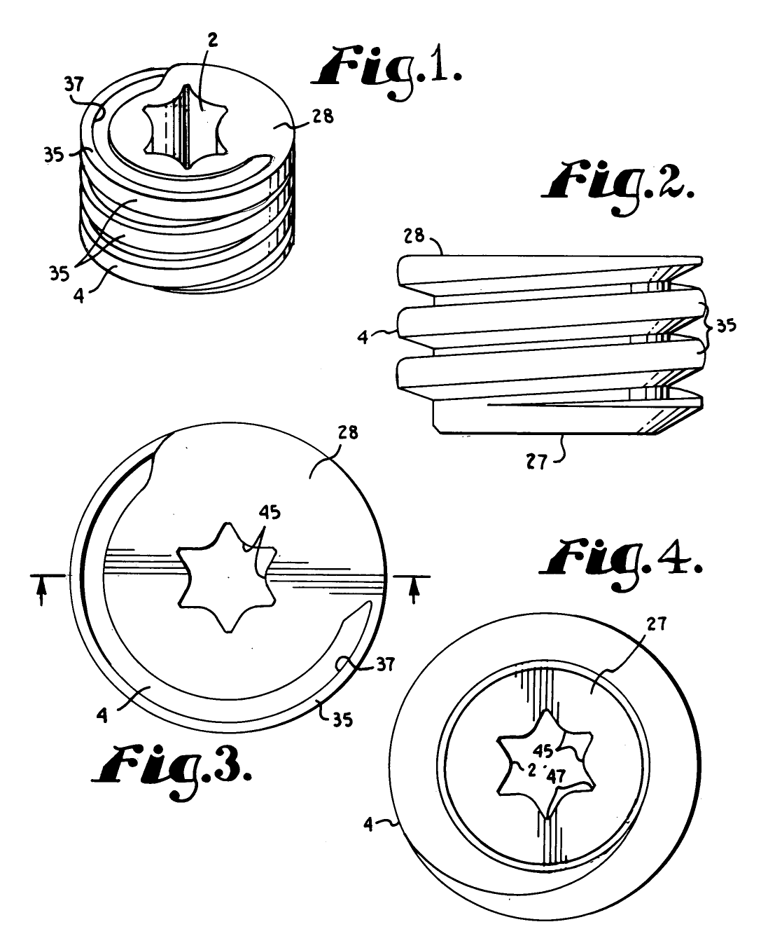

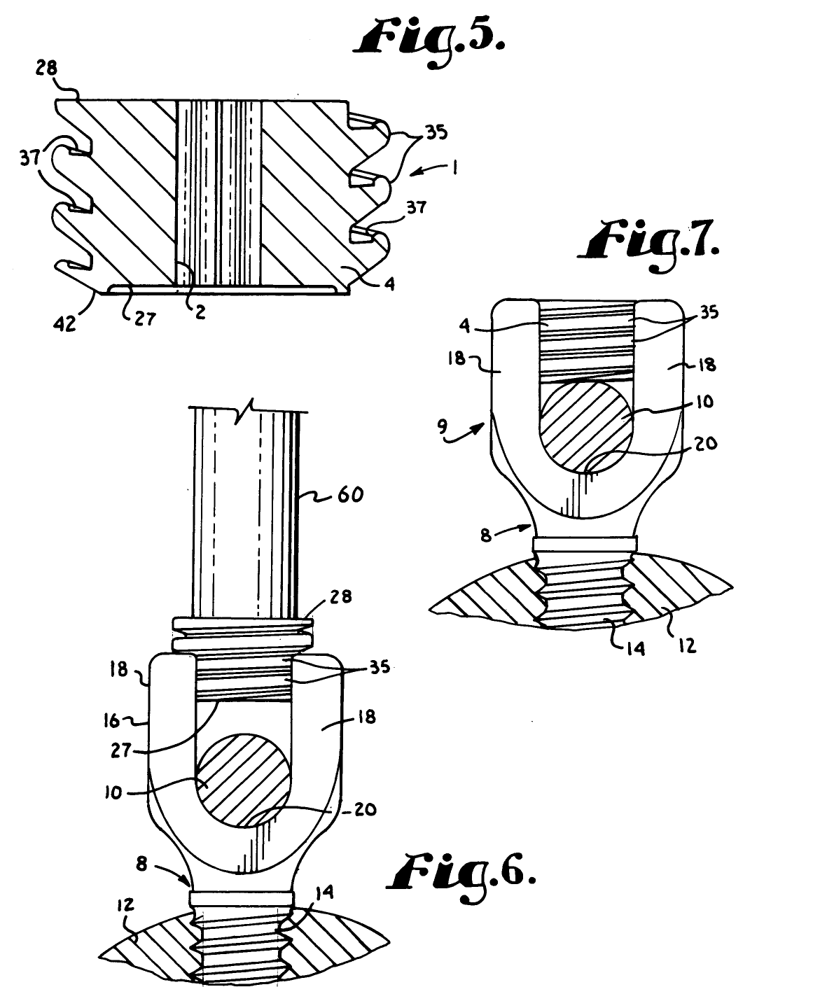

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

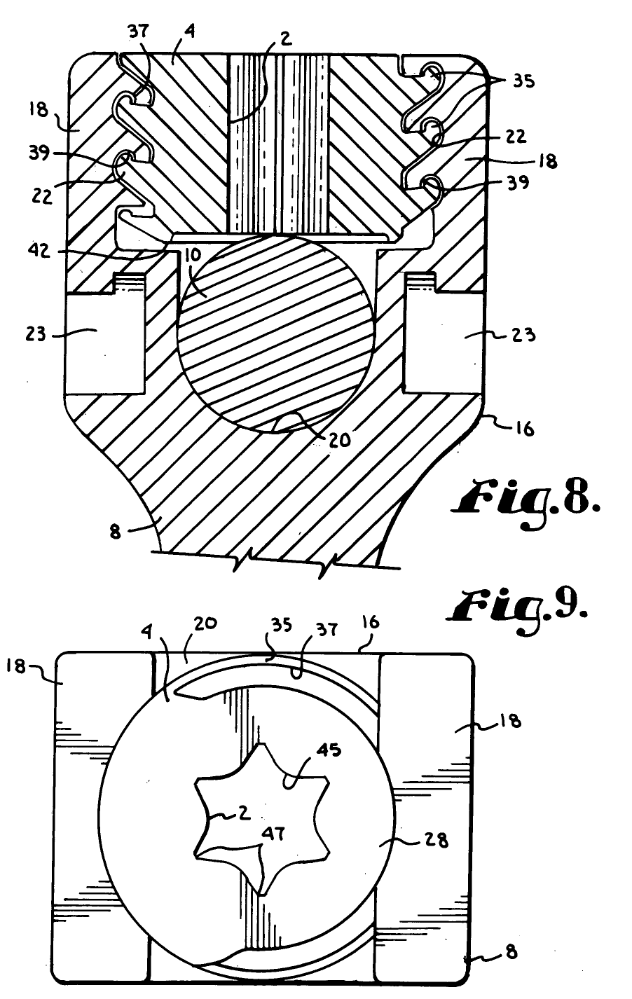

Referring to the drawings in more detail, the reference numeral 1 generally designates an anti-splay closure with a multi-surfaced aperture, such as a multi-lobular or curved surface aperture 2. The closure 1 generally includes a body 4 that is used in cooperation with an open headed bone implant screw 8 (FIGS. 6 and 7) to form an implant anchor assembly 9 to secure or anchor a spinal fixation member or rod 10 with respect to a bone 12, such as a vertebra.

The bone screw 8 includes a threaded shank 14 for threadably implanting into the bone 12 and an open head 16 formed by a pair of spaced apart arms 18 defining a U-shaped channel 20 therebetween to receive the rod 10. Inner and facing surfaces of the arms 18 have internal mating grooves or guide and advancement structures 22 (FIG. 8) tapped, or otherwise formed, therein. The head 16 has tool grip indentations 23 (FIG. 8) that allow a gripping tool (not shown) to securely hold the head 16 and facilitate gripping the bone screw 8 during manipulation for implantation of the bone screw 8 into the bone 12.

The body 4 is cylindrical in external shape about an axis of rotation 25 (FIG. 7) and has a forward, leading, or inner end 27 and a rear, trailing, or outer end 28.

The body 4 is provided with a guide and advancement flange 35 which extends helically about the cylindrical closure body 4. The flange 35 is enlarged near an outer periphery or radial crest thereof to form a generally inwardly facing or inward anti-splay surface 37. In a similar manner, the mating guide and advancement structures 22 are enlarged near the radially outward peripheries thereof to form generally outwardly facing or outward anti-splay surfaces 39. The anti-splay or splay resisting surfaces 37 and 39 mutually engage or slide closely to one another when the body 4 is rotated and thereby the body 4 is advanced into the bone screw head 16 so as to interlock thereby also interlocking the body 4 to the arms 18 to resist or prevent outward splaying of the arms 18 in reaction to torque or other forces.

Although particular contours of the flange 35 and mating structures 22 are shown herein, other contours of anti-splay guide and advancement flanges 35 and mating structures 22 are foreseen. Examples of such alternative configurations of anti-splay or splay resisting guide and advancement flange and mating structures are disclosed in U.S. patent application Ser. No. 10/236,123 which is now U.S. Pat. No. ______ , and which is incorporated herein by reference. The flange 35 and structures 22 cooperate to guide and advance the body 4 into clamping engagement with the rod 10 within the channel 20 in response to clockwise rotation of the body 4.

In order to more positively secure the rod 10 within the head 16 of the bone screw 8, the body 4 is provided with a V-ring or “cup point” 42 on the inner or forward end 27 thereof. The V-ring 42 cuts into the surface of the rod 10 when the body 4 is tightly torqued into the head 16. The V-ring 42 extends about a periphery of the inner end 27 of the body 4 and, thus, provides two possible areas of engagement between the body 4 and the rod 10.

In the great majority of cases, the body 4 is torqued into engagement with the rod 10 in the bone screw 8 and the anchor assembly 9 is permanently implanted in the bone 12. However, spinal alignment geometry is complex and it is sometimes necessary to make adjustments to a spinal fixation system. Additionally, slippage or failure of spinal fixation components can occur due to injury to the patient, deterioration of bone tissue, or the like. It is also possible that an implant system using anchored rods might be used therapeutically, for example, to set a broken bone, and subsequently removed. For these reasons, implant anchor assemblies often provide structures or mechanisms for releasing an anchor assembly 9 to make such adjustments or changes in a spinal fixation system. The anchor assembly 9 of the present invention provides the aperture 2 not only for installation but also for engaging the body 4 to retract it out of the bone screw head 16 to release the rod 10 to enable adjustment of the position of the rod 10 relative to the bone screw 8.

In particular, the multi-surfaced aperture 2 is coaxially positioned relative to the body 4 axis of rotation 25 and provided for non-slip engagement by an insertion tool and the same or a different closure removal tool (not shown) having a body with a shape which is complementary to the shape of the socket formed by the aperture 2 and an outwardly extending handle, normally of a type conventionally known as a “torx” driver. The illustrated aperture 2 is multi-lobular and is formed by a plurality of circumferentially spaced, axially extending lobes 45 separated by intervening spline receiving grooves 47. The closure installation and removal tool 60 for use with the aperture 2 has a lower portion with a shape which is complementary to the socket formed by the aperture 2 and includes circumferentially spaced splines corresponding to the grooves 47 and removal and installation tool 60 grooves corresponding to the lobes 45. The aperture 2 may be of a Torx type shape which is “hexlobular” or six lobed, or other multi-lobular shape, such as “penta-lobular” or five lobed, etc.

The axis 25 passes through the aperture 2 so as to facilitate rotation of the body 4 by a tool having a single mating projection that conforms to the aperture 2.

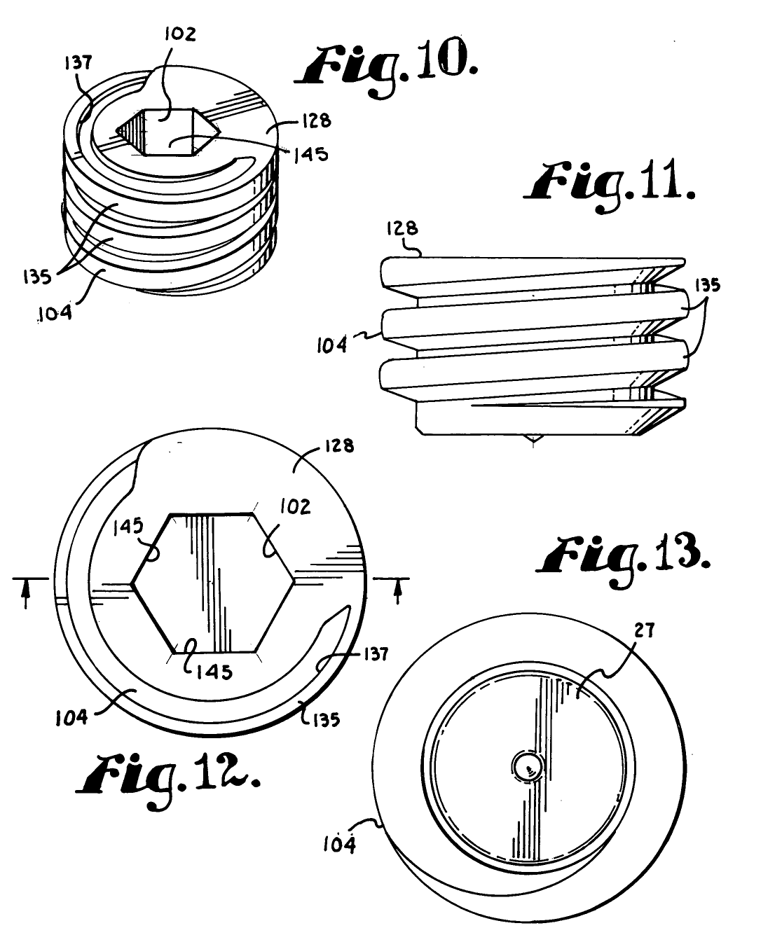

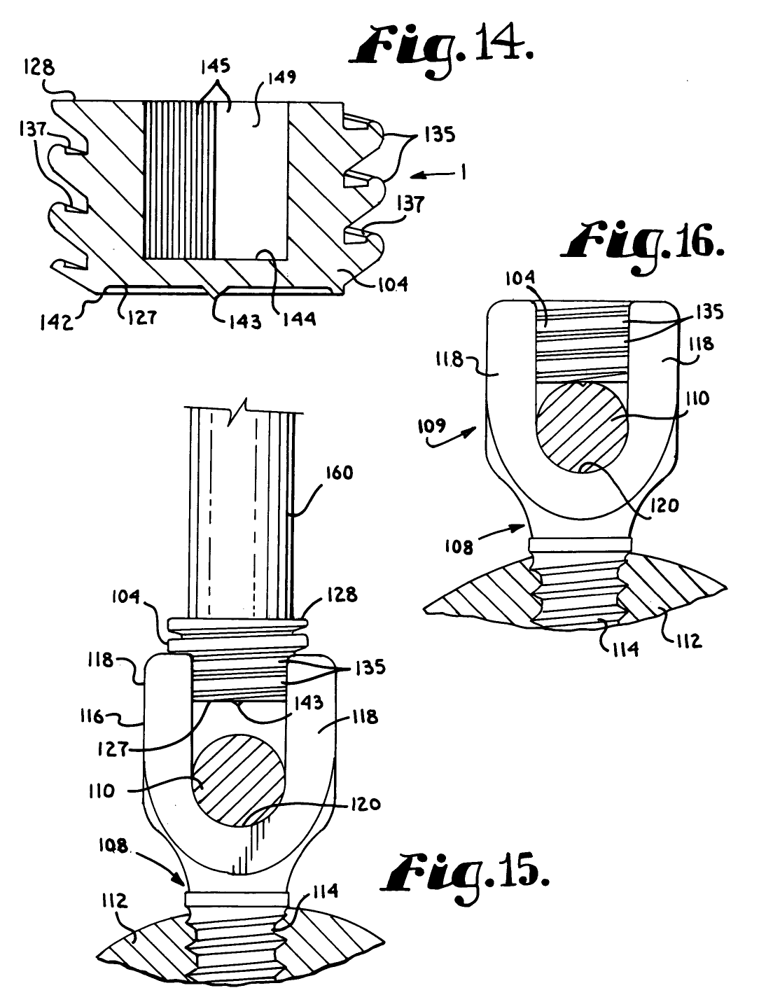

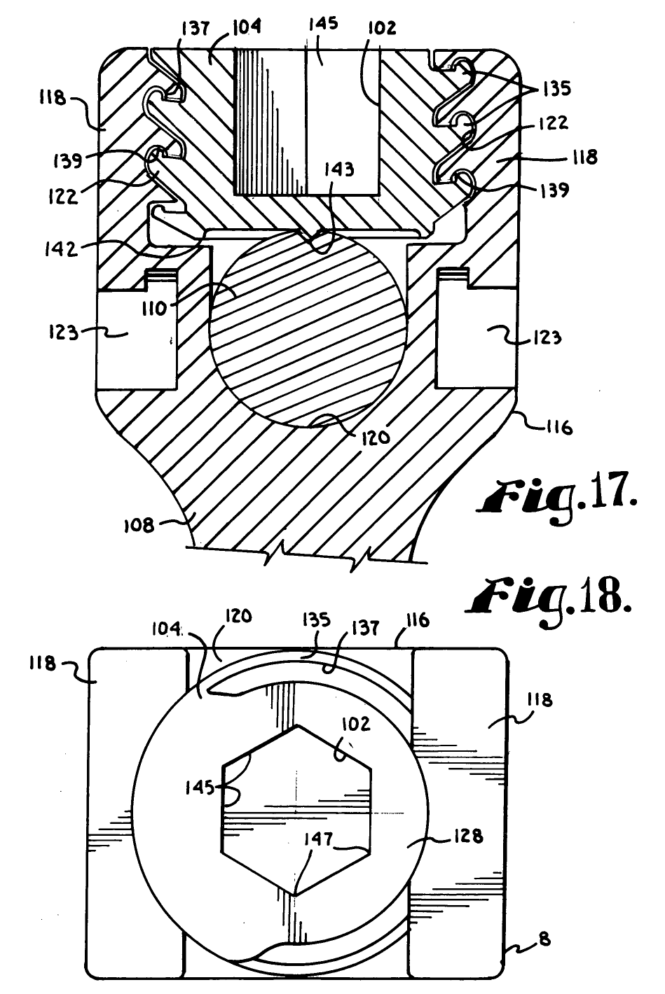

Illustrated in FIGS. 10 to 18 is a second embodiment of an anti-splay closure generally identified by the referenced numeral 101 with a multi-surfaced aperture 102. The closure 101 generally includes a body 104 that is used in cooperation with an open headed bone implant screw 108 (FIGS. 15 and 16) to form an implant anchor assembly 109 to secure or anchor a spinal fixation member or rod 110 with respect to a bone 112, such as a vertebra.

The bone screw 108 includes a threaded shank 114 for threadably implanting into the bone 112 and an open head 116 formed by a pair of spaced apart arms 118 defining a U-shaped channel 120 therebetween to receive the rod 110. Inner and facing surfaces of the arms 118 have internal mating grooves or guide and advancement structures 122 (FIG. 17) tapped, machined by single-point tooling techniques or otherwise formed, therein. The head 116 has grip indentations 123 (FIG. 17) to facilitate gripping the bone screw 108 by an appropriate screw gripping tool (not shown) during manipulation for implantation of the bone screw 108 into the bone 112.

The body 104 is cylindrical in external shape about an axis of rotation 125 (FIG. 16) and has a forward, leading, or inner end 127 and a rear, trailing, or outer end 128.

The body 104 is provided with a guide and advancement flange 135 which extends helically about the cylindrical closure body 104. The flange 135 is enlarged near an outer periphery or radial crest thereof to form a generally inwardly facing or inward anti-splay surface 137. In a similar manner, the mating guide and advancement structures 122 are enlarged near the radially outward peripheries thereof to form generally outwardly facing or outward anti-splay surfaces 139. The anti-splay or splay resisting surfaces 137 and 139 mutually engage when the body 104 is rotated and advanced into the bone screw head 116, so as to interlock thereby also interlocking the body 104 to the arms 118 to resist outward splaying of the arms 118 in reaction to torque or other forces subsequently subjected to the implant.

Although particular contours of the flange 135 and mating structures 122 are shown herein, other contours of anti-splay guide and advancement flanges 135 and mating structures 122 are foreseen. Examples of such alternative configurations of anti-splay or splay resisting guide and advancement flange and mating structures are disclosed in U.S. patent application Ser. No. 10/236,123 which is now U.S. Pat. No. ______ , which is incorporated herein by reference. The flange 135 and or mating structures 122 cooperate to guide and advance the body 104 into clamping engagement with the rod 10 within the channel 120 in response to rotation of the body 104.

In order to more positively secure the rod 110 within the head 116 of the bone screw 108, the body 104 is provided with a V-ring or “cup point” 142 on the inner or forward end 127 thereof. The V-ring 142 cuts into the surface of the rod 110 when the body 104 is tightly torqued into the head 116. The V-ring 142 extends about a periphery of the inner end 127 of the body 104 and, thus, provides two possible areas of engagement between the body 104 and the rod 110. Centrally located relative to the V-ring 142 and coaxially extending from the body forward end 127 is a point 143 for penetrating into the rod 110.

In the great majority of cases, the body 104 is torqued into engagement with a rod 110 in a bone screw 108 and the anchor assembly 109 is thereafter permanently implanted in the bone 112. However, spinal alignment geometry is complex, and it is sometimes necessary to make adjustments to a spinal fixation system. Additionally, slippage or failure of spinal fixation components can occur due to injury to the patient, deterioration of bone tissue, or the like. It is also possible that an implant system using anchored rods might be used therapeutically, for example, to set a broken bone, and subsequently removed. For these reasons, implant anchor assemblies often provide structures or mechanisms for releasing an anchor assembly 109 to make such adjustments or changes in a spinal fixation system. The anchor assembly 109 of the present invention provides removing the body 104 to retract it out of the bone screw head 116 so as to release the rod 110 and enable adjustment of the position of the rod 110 relative to the bone screw 108.

In the present embodiment, the removal structure is the same as the installation structure. In particular, the multi-surfaced aperture 102 is used for both insertion and removal. The aperture 102 is coaxially positioned relative to the body 104 axis of rotation 125 and provided for non-slip engagement by a closure installation and removal tool 160 having a shape which is complementary to the shape of the aperture 102. The aperture 102 does not fully penetrate from the rear end 128 to the front end 127, but rather is spaced therefrom by a wall 144. The illustrated aperture 102 is multi-surfaced and is formed by a plurality of circumferentially spaced, axially planar surfaces 145 joined at edges 147. The closure removal tool 160 for engagement with the aperture 102 has a body with a shape which is complementary thereto and fits in a hexagonal shaped socket 149 formed by the walls of the aperture 102. The illustrated aperture 102 has what is normally referred to as an Allen configuration.

It is also foreseen that the multi-surfaced aperture 102 could be of other shapes, such as a multi-faceted shape having a square, triangular, rectangular, etc. shape. Alternatively, other non-circular, multi-surfaced shapes are envisioned for the shape of the aperture 102; however, the axis 125 passes through the aperture 102 so as to facilitate rotation of the body 104 by a tool having a single mating projection that conforms to the aperture socket 149.

It is to be understood that while certain forms of the present invention have been illustrated and described herein, it is not to be limited to the specific forms or arrangement of parts described and shown.

What is claimed and desired to be secured by Letters Patent is as follows:

1. A closure for setting engagement with a structural member and comprising:
(a) a substantially cylindrical body having an outer cylindrical surface relative to a central closure axis;
(b) a substantially continuous guide and advancement flange extending helically about said outer cylindrical surface, said flange having a leading surface and a trailing surface relative to a direction of forward advancement;
(c) at least one of said leading surface or said trailing surface being compound in contour and including an inward facing anti-splay surface component facing generally toward said closure axis; and
(d) said body having a multi-surface aperture formed therein that is aligned with said closure axis and that is elongated along said closure axis, said aperture opening onto a trailing surface of said body and including a plurality of circumferentially spaced, centrally facing surfaces extending substantially parallel to said closure axis that are aligned to form a removal socket adapted to receive a removal tool.

2. The closure as set forth in claim 1 wherein said multi-surfaced aperture includes:
(a) a multi-lobular aperture elongated along said closure axis, said aperture including a plurality of circumferentially spaced lobes extending substantially parallel to said closure axis and facing generally toward said closure axis.

3. The closure as set forth in claim 2 wherein:
(a) said lobes circumferentially alternate with grooves extending substantially parallel to said closure axis.

4. The closure as set forth in claim 1 and including:
(a) said body having a forward end relative to said forward advancement direction; and
(b) said body having a V-shaped set ring formed on said forward end to enhance setting engagement of said body into a surface of a structural member.

5. The closure as set forth in claim 1 in combination with a bone implant screw adapted for connection to a bone fixation structural member, said bone implant screw including:
(a) a threaded shank adapted for threaded implanting into a bone;
(b) an open head formed by a pair of spaced apart arms having mutually facing channel surfaces defining a structural member receiving channel to receive a bone fixation structural member; and
(c) said mutually facing channel surfaces having respective mating guide and advancement structures formed therein which are compatible with and rotatably mateable with said guide and advancement flange to enable guiding and advancement of said body into said channel to thereby clamp said bone fixation structural member therein and to interlock said body and arms.

6. The closure and bone implant screw combination as set forth in claim 6 wherein:
(a) said mating guide and advancement structures of said bone implant screw include an outward anti-splay surface component which cooperates with said inward anti-splay surface component of said closure in such a manner as to resist a tendency of said arms to splay in reaction to torquing said closure into engagement with said bone fixation structural member.

7. The combination as set forth in claim 6 wherein:
(a) said guide and advancement flange has a relatively enlarged region near an outer periphery thereof that forms said inward anti-splay surface component;
(b) said mating guide and advancement structures are contoured in a complementary manner to said guide and advancement flange to form said outward anti-splay surface component; and
(c) said inward anti-splay surface component engages said outward anti-splay surface component when said closure is guided and advanced into said open screw head of said bone implant screw so as to interlock said body to said arms to resist radially outward splaying movement of said arms.

8. The closure according to claim 1 wherein:
(a) said multi-surfaced aperture is hexagonal in cross section.

9. The closure according to claim 1 wherein:
(a) said multi-surfaced aperture passes entirely through said body from said trailing surface to said leading surface.

10. The closure according to claim 1 wherein:
(a) said multi-surfaced aperture passes only partially through said body from said trailing surface.

11. A closure for setting engagement with a structural member and comprising:
(a) a substantially cylindrical body having an outer cylindrical surface relative to a central closure axis;
(b) a guide and advancement flange extending helically about said outer cylindrical surface, said flange having a trailing surface relative to said forward advancement direction;
(c) said trailing surface being compound in contour and including an inward facing anti-splay surface component facing generally toward said closure axis; and
(d) said body having a multi-lobular aperture formed therein which is aligned on and elongated along said closure axis, said aperture including a plurality of circumferentially spaced lobes extending substantially parallel to said closure axis and said lobes circumferentially alternating with bore grooves extending substantially parallel to said closure axis to form a removal socket adapted to receive a removal tool.

12. The closure as set forth in claim 11 and including:
(a) said body having a forward end relative to a direction of forward advancement; and
(b) said body having a V-shaped set ring formed on said forward end to enhance setting engagement of said body into a surface of a structural member.

13. The closure as set forth in claim 11 in combination with a bone implant screw adapted for connection to a bone fixation structural member, said bone implant screw including:
(a) a threaded shank adapted for threaded implanting into a bone;
(b) an open head formed by a pair of spaced apart arms having mutually facing channel surfaces defining a structural member receiving channel to receive a bone fixation structural member; and
(c) said mutually facing channel surfaces having an internal mating guide and advancement structures formed therein which are compatible for slidably mating with said flange upon rotation of said body to enable advancement of said body into said channel to thereby clamp said bone fixation structural member therein and to interlock said body to said arms to resist splaying of said arms.

14. The closure and bone implant screw combination as set forth in claim 13 wherein:
(a) said mating guide and advancement structures of said bone implant screw include an outward anti-splay surface component which cooperates with said inward anti-splay surface component of said flange in such a manner as to resist splaying of said arms.

15. The combination as set forth in claim 14 wherein:
(a) said flange has a relatively enlarged region near an outer periphery thereof that forms said inward anti-splay surface component;
(b) said mating guide and advancement structures are contoured in a complementary manner to said flange to form said outward anti-splay surface component; and
(c) said inward anti-splay surface component engages said outward anti-splay surface component when said closure is rotated into said open screw head of said bone implant screw.

16. A closure for setting engagement with a structural member and including a substantially cylindrical body having an outer cylindrical surface relative to a central closure axis and a substantially continuous guide and advancement flange extending helically about said outer cylindrical surface, said flange having a leading surface and a trailing surface relative to a direction of forward advancement, the improvement comprising:
(a) at least one of said leading surface and said trailing surface being compound in contour and including an inward facing anti-splay surface component facing generally toward said closure axis; and
(b) said body having a multi-surfaced aperture formed therein which is located and elongated along said closure axis, said aperture including a plurality of circumferentially spaced surfaces extending substantially parallel to said closure axis so as to form a removal socket adapted to receive a removal tool.

17. The closure as set forth in claim 16 wherein said multi-surfaced aperture is:
(a) a multi-lobular aperture elongated along said closure axis, said aperture including a plurality of circumferentially spaced lobes extending substantially parallel to said closure axis and said lobes circumferentially alternating with grooves extending substantially parallel to said closure axis.

18. The closure as set forth in claim 16 and including:
(a) said body having a forward end relative to said direction of forward advancement; and
(b) said body having a V-shaped set ring formed on said forward end to enhance setting engagement of said body into a surface of such a structural member.

19. The closure as set forth in claim 16 in combination with a bone implant screw adapted for connection to a bone fixation structural member, said bone implant screw including:
(a) a threaded shank adapted for threaded implanting into a bone;
(b) an open head formed by a pair of spaced apart arms having mutually facing channel surfaces defining a structural member receiving channel to receive a bone fixation structural member;
(c) said mutually facing channel surfaces having respective mating guide and advancement structures formed therein which are compatible to allow rotational mating with said guide and advancement flange to enable guiding and advancement of said body into said channel to thereby clamp said bone fixation structural member therein and to interlock said arms to said body to resist splaying of said arms; and
(d) said mating guide and advancement structures of said bone implant screw including an outward anti-splay surface component which cooperates with said inward anti-splay surface component of said flange in such a manner as to resist a tendency of said arms to splay in reaction to torquing and other forces.

20. The combination as set forth in claim 19 wherein:
(a) said guide and advancement flange has a relatively enlarged region near an outer periphery thereof that forms said inward anti-splay surface component;
(b) said mating guide and advancement structures are contoured in a complementary manner to said guide and advancement flange to form said outward anti-splay surface component; and
(c) said inward anti-splay surface component engages said outward anti-splay surface component when said closure is guided and advanced into said open screw head of said bone implant screw so as to radially interlock.
